(19) United States
(12) Patent Application Publication
(10) Pub. No.: US 2012/0302530 A1
(43) Pub. Date: Nov. 29, 2012

(54) 23-HYDROXY-BETULINIC ACID DERIVATIVES, PREPARATION METHODS AND USES THEREOF

(75) Inventors: Nan Zhang, Nanjing (CN); Rong Zhong, Nanjing (CN)

(73) Assignee: SUZHOU MAIDIXIAN PHARMACEUTICAL INC., Suzhou, JS (CN)

(21) Appl. No.: 13/511,399

(22) PCT Filed: May 13, 2010
(86) PCT No.: PCT/CN2010/072740

§ 371 Date: Aug. 9, 2012

(30) Foreign Application Priority Data
Nov. 23, 2009 (CN) 200910186536.6

(51) Int. Cl. A61K 31/568; A61P 31/18; A61P 35/00; C07J 53/00

(52) U.S. Cl. 514/169; 552/510

(57) ABSTRACT

The present invention discloses 23-hydroxyl-betulinic acid derivatives, including 3-oxo-23-hydroxyl-betulinic acid, 3-oxo-23-hydroxyl-dihydrobetulinic acid, and 23-hydroxyl-dihydrobetulinic acid. The present invention also discloses the preparation methods thereof and medical applications as anti-tumor and anti-HIV agents.

### CROSS-REFERENCE TO RELATED APPLICATIONS

This application is a national stage application under 35 U.S.C. 371 of International Patent Application Serial No. PCT/CN2010/072740, entitled “23-HYDROXY-BETULINIC ACID DERIVATIVES, PREPARATION METHODS AND USES THEREOF,” filed May 13, 2010, which claims priority from Chinese Patent Application No. 200910186536.6, filed Nov. 23, 2009.

### FIELD OF INVENTION

This invention relates to 23-hydroxy-betulinic acid derivatives having anti-tumour and anti-aids effects, and preparation methods and uses thereof.

### BACKGROUND

Betulinic acid, also called betulic acid, is a pentacyclic triterpenoid, with English name as “Betulinic acid”, chemical name as 3β-hydroxy-1up-20(29)-ene-28-oic acid, molecular formula as C₃₀H₄₈O₃, and molecular weight as 456.71. Betulinic acid has several biologic sources, e.g., it can be extracted from recrystallizing ethanolic extract of syzygium jambos leaves or cortices of white birch; and it can also be obtained with chemosynthesis by using betulin as the raw material. It can be used as aromatizing agent. Betulinic acid is a colorless crystal, with melting point as 316˜318° C., optical rotation as [α]_{D}+8°, and can be dissolved in ether, ethanol, acetone, chloroform, and pyridine. The properties and functions of betulinic acid can be found in the following three arts: ZHOU, Yingxia, et al., RESEARCH ON BETULINIC ACID AND ITS DERIVATIVES, Issue 6, 2005, China Pharmaceutical Magazine; Xu, Ping, et al., ADVANCES IN RESEARCH ON ANTITUMOR ACTIVITY OF BETULINIC ACID COMPOUND, Issue 2, 2006, China Apothecary; and Li, Dan, et al., ADVANCES IN RESEARCH ON BETULINIC ACID AND ITS DERIVATIVES, Issue 3, 2004, Advance in Pharmacy. Betulinic acid has a number of biological activities such as antitumor, anti- HIV, anti-inflammatory and anti-malaria in vitro, and has certain selective cytotoxicity on tumor cells and better therapeutic index, wherein there is no obvious toxic reaction when even reaching 500 mg/kg. In mid 1990s, it was found that betulinic acid can selectively kill the human melanomatous cell without killing healthy cells. Recent study discloses that betulinic acid also has inhibiting effects to malignant cells such as cerebroma, neuroectodermal tumor, leukemia and the like. The betunlinic acid anti-tumor mechanism of action is known as reducing tumor cell mitochondria membrane potential to change the permeability of mitochondria in cells, and finally to lead to apoptosis; and is related to up-regulate expression of tumor cytomembrane p53 protein, and change tumor cytomembrane Ca²⁺, Mg²⁺ and -ATP enzymatic activities. See the below two documents for details: Y E, Yinying, et al., RESEARCH ON ANTI-MELANOMA EFFECT IN VITRO AND IN VIVO OF 23-HYDROXYL-BETULIC ACID [J]., Issue 1, 2000, China Tumor Clinic and Rehabilitation; and CHENG, Yan, et al., LoVo APOPTOSIS AND CHANGES IN MITOCHONDRIAL MEMBRANE POTENTIAL INDUCED BY 23-HYDROXYL-BETULIC ACID [J]., Issue 3, 2007, ACTA PHARMACOLOGICA SINICA.

The traditional Chinese medicine—pulsatilla chinensis has efficacies of clearing away heat and toxic material and promoting blood circulation by removing blood stasis, and is clinically applied to treat tumors of intestine, such as colon cancer, rectal cancer, as well as cervical cancer, pituitary adenoma, thyrophyma, and lung cancer, see Ye w c, Zhang Q W, Hsiao W L, et al., New lupane glycosides from pulsatila chinensis [J]. P/anta Medwa, 2002, 68: 183-6. 23-hydroxyl-betulic acid is a triteroene found from pulsatilla chinensis, which has a content up to 2.5%, see Y E, Yinying, H E Daowei, Y E Wencai, et al., RESEARCH ON ANTI-MELANOMA EFFECT IN VITRO AND IN VIVO OF 23-HYDROXYL-BETULIC ACID [J]., China Tumor Clinic and Rehabilitation, 2000 7 (1): 5-7. Recent research has discovered that 23-hydroxyl-betulic acid can selectively inhibit propagation of melanoma cell B16 of mice, the mechanism of action of which is mainly inducing apoptosis of B16 cell, see the below two documents: Ye Y Y, He D W, Ye W C, et al., The study of 23-hydroxybetulinic Acid against melanoma in vivo and in vitro [J]. Chin J Clin Oncol Rehab. 2000, 7 (1): 5-7; Wu W K, Ho J C, Cheung F W, et al. Apoptotic activity of betulinic acid derivatives on murine melanoma B16 cell line [J] Eur J Pharmacol. 2004, 498 (1-3): 71-8.

From above, betulinic acid and 23-hydroxyl-betulinic acid have antitumor effects, and thus can be used as an antineoplastic curative. Currently, when the antineoplastic drug is applied to human body, there are big differences in the drug action of different individuals, and the molecular mechanism of action of antineoplastic drug has not been thoroughly studied. Thus, different individuals can only select drugs that have great drug action and less side effects from various antineoplastic drugs. There is a desire to develop more antineoplastic drugs or compounds having antineoplastic activities. In view of the antineoplastic effects of betulinic acid and 23-hydroxyl-betulinic acid, it is expected to find other compounds having biological activities from derivatives of betulinic acid and 23-hydroxyl-betulinic acid. These compounds can be used in antineoplastic curative, or have other effects such as anti-HIV/AIDS.

### SUMMARY

In order to achieve the above goals, the inventors have conducted researches on many derivatives of betulinic acid or 23-hydroxyl-betulinic acid, and found that three kinds of 23-hydroxyl-betulinic acid derivatives have antitumor and anti-HIV/AIDS activities. This leads to the present invention. The first objective of the present invention is to provide a new compound having antitumor and anti-HIV/AIDS effects. The new compound belongs to a derivative of 23-hydroxyl-betulinic acid. The second objective of the present invention is to provide a method for preparing the new compound; and the third objective of the present invention is to describe use of the new compound.

The present invention provides three kinds of new derivatives of 23-hydroxyl-betulinic acid, with the chemical names as 3-carboxyl-23-hydroxyl-betulinic acid, 3-carboxyl-23-hydroxyl-dihydrobetulinic acid, and 23-hydroxyl-dihydrobetulinic acid, respectively, and chemical formula as follows:

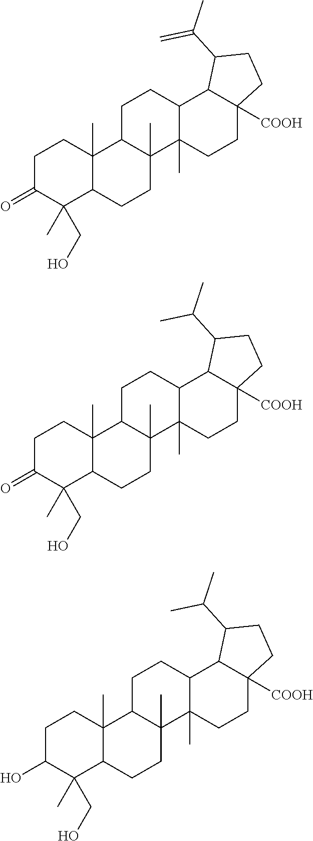

3-carboxyl-23-hydroxyl-betulinic acid (also named 3-carbonyl-23-hydroxy-betulinic acid) has molecular formula as C₃₀H₄₆O₄, molecular weight as 470.68, and is prepared as follows:

(1) 500 mg 23-hydroxyl-betulinic acid is dissolved in 1 ml pyridine, stirred to be uniform under −5° C., slowly dropped 0.15 ml acetic anhydride therein, and then stirred for reaction under −5° C. for about 24 hours. At this temperature, ammonia chloride saturated solution is slowly dropped until there is no bubble generated. Upon three times extraction of ethyl acetate, the organic layer is merged, saturated sodium chloride solution is used to wash the organic layer twice, and then anhydrous sodium sulfate is used to dry out. Upon the organic solvent ethyl acetate is spin dry, column separation is used. Besides recycling part of the material, the compounds 3, 23-diacetyl-23-hydroxyl-betulinic acid (defined as zhangnan-1), 23-diacetyl-23-hydroxyl-betulinic acid (defined as zhangnan-2), and 3-diacetyl-23-hydroxyl-betulinic acid (defined as zhangnan-6) are obtained, respectively. The compounds zhangnan-1, zhangnan-2, and zhangnan-6 account for 30%, 60% and 10% of the total products, respectively. The total yield of this reaction step is 65%, and the reaction formula is as follows:

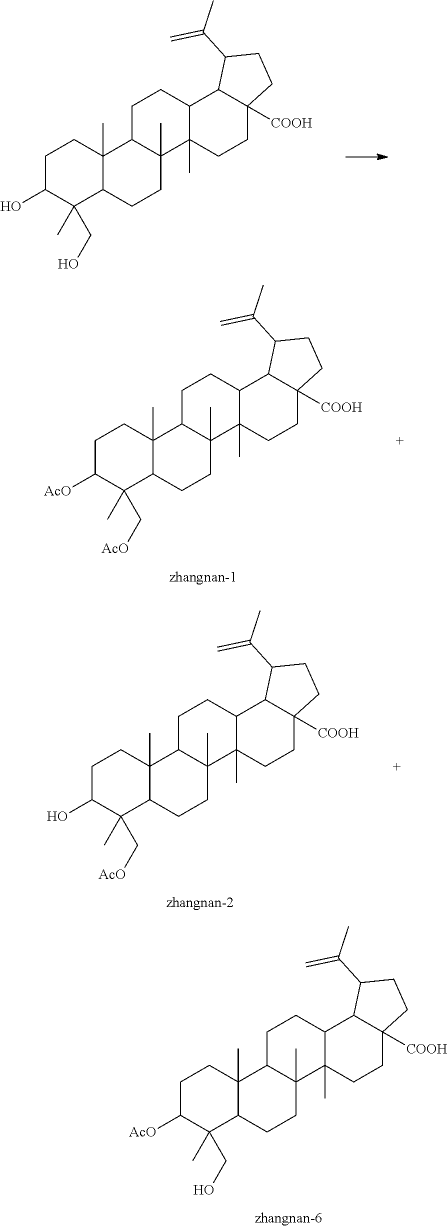

(2) 100 mg of the compound zhangnan-2 is dissolved and dispersed in 30 ml methylene chloride. Upon oxygenant dicyclohexylcarbodiimide (58 mg) is added under ice-bath, stirring reaction is implemented under room temperature for 4 hours, and then the solvent methylene chloride is spin dry, dissolved with ethyl acetate and conducted column separation with coarse silica gel. The compound of 3-carboxyl-23-diacetyl-23-hydroxyl-betulinic acid (defined as zhangnan-4) (70 mg) is obtained. The yield of this reaction step is 80%, and the reaction formula is as follows:

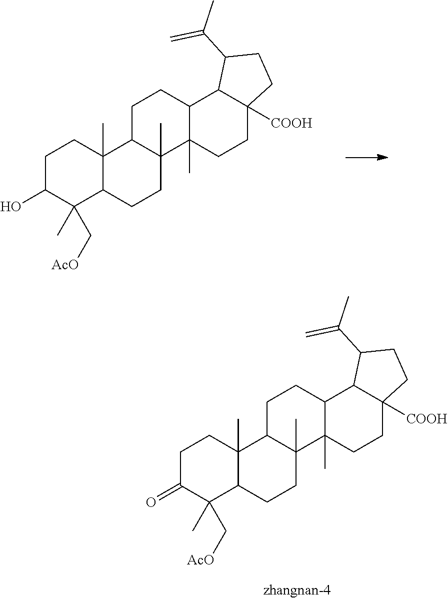

(3) 50 mg of the compound zhangnan-4 is taken to disperse and dissolve in 30 ml methanol. Upon adding 5 ml of saturated potassium carbonate solution, stirring reaction is conducted under room temperature for 2 hours. Upon three times extraction of ethyl acetate, the organic layers are merged. The saturated sodium chloride solution is used to wash the organic layers twice, and then anhydrous sodium sulfate is used to dry out. Upon the organic solvent ethyl acetate is spin dry, column separation is conducted. The compound of 3-carboxyl-23-hydroxyl-betulinic acid (defined as zhangnan-5) (40 mg) is obtained. The yield of this reaction step is 90%, and the reaction formula is as follows:

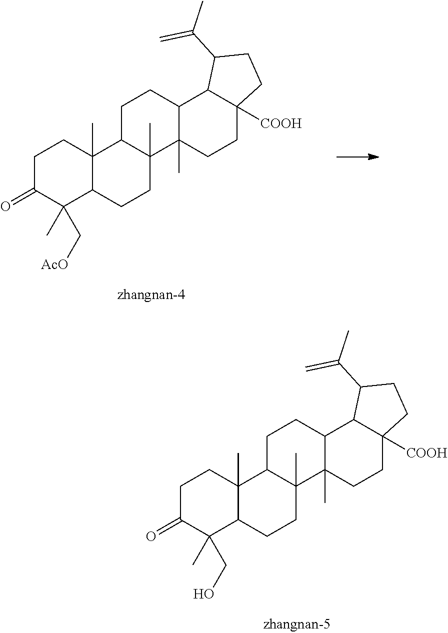

Compound zhangnan-5

H-NMR (400 MHz, CDCl3) δ: 0.963, 0.969, 0.992, 1.015, 1.690 (each 3H, s, 5×CH3), 2.99 (1H, m, H-19), 4.71 (1H, d, J=1.6 Hz, 29-H), 4.61 (1H, d, J=1.6 Hz, 29-H).

C-NMR (400 MHz, CDCl3) δ: 213.95, 182.21, 150.31, 109.79, 56.36, 53.30, 49.20, 48.17, 46.93, 44.58, 42.56, 40.38, 40.34, 38.41, 37.43, 37.04, 36.79, 33.08, 32.13, 30.56, 29.60, 25.46, 22.18, 21.51, 19.34, 15.89, 14.52, 13.54.

### MS (ESI): m/z 440 [M−2O]⁻.

3-carboxyl-23-hydroxyl-dihydrobetulinic acid (also called 3-carbonyl-23-hydroxy-dihydrobetulinic acid) has molecular formula as C₃₀H₄₈O₄, molecular weight as 472.7, and is prepared as follows:

30 mg of the compound zhangnan-5 is taken to disperse and dissolve in 30 ml methanol. Upon adding about 10 mg of reductant Pd/C, the reaction system is sealed. Vacuum is drawn, hydrogen is filled, and stirring reaction in the hydrogen flow is conducted under room temperature for 10 hours. After the reaction, the Pd/C is filtered out and then the solvent is spun dry, to obtain 25 mg of the product 3-carboxyl-23-hydroxyl-dihydrobetulinic acid (defined as zhangnan-7). The yield of this reaction is 85%, and the reaction formula is as follows:

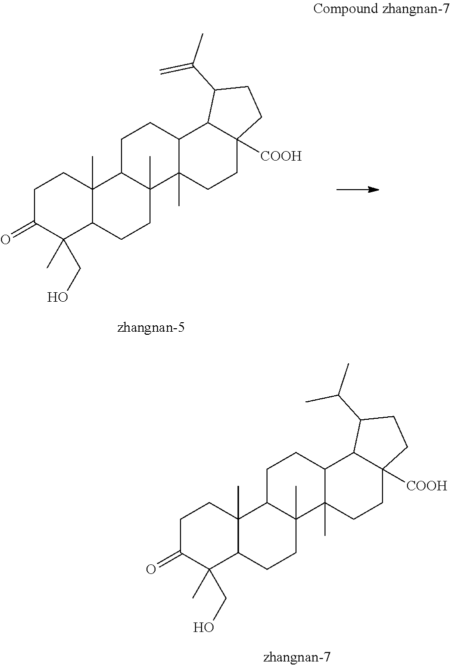

H-NMR (400 MHz, CDCl3) δ: 0.755 (3H, d, J=7.2 Hz), 0.963 (3H, d, J-7.2 Hz), 0.894, 0.936, 1.048, 1.301 (each 3H, s, 4×CH3), 2.243 (4H, m).

### C-NMR (400 MHz, CDCl3) δ: 212.69, 177.79, 56.29, 52.98, 48.52, 47.65, 44.19, 44.12, 42.71,37.87, 37.33, 37.22, 36.78, 33.16, 32.12, 29.82, 29.59, 27.02, 23.45, 22.93, 22.19, 21.59, 16.12, 14.99, 14.58, 13.63, 12.04.

### MS(ESI): m/z 442 [M−2OH]⁻_{∘}

23-hydroxyl-dihydrobetulinic acid (also called 3-hydroxy-dihydrobetulinic acid) has molecular formula as C₃₀H₄₀O₄, molecular weight as 474.72, and is prepared as follows:

30 mg of the compound 23-hydroxyl-betulinic acid is taken to disperse and dissolve in 30 ml methanol. Upon adding about 10 mg reductant Pd/C, the reaction system is sealed. Vacuum is drawn, hydrogen is filled, and stirring reaction in the hydrogen flow is conducted under room temperature for 10 hours. After the reaction is over, the Pd/C is filtered out, and then the solvent is spun dry, to obtain 20 mg of the product 23-hydroxyl-dihydrobetulinic acid (defined as zhangnan-8). The yield of this reaction is 70%, and the reaction formula is as follows:

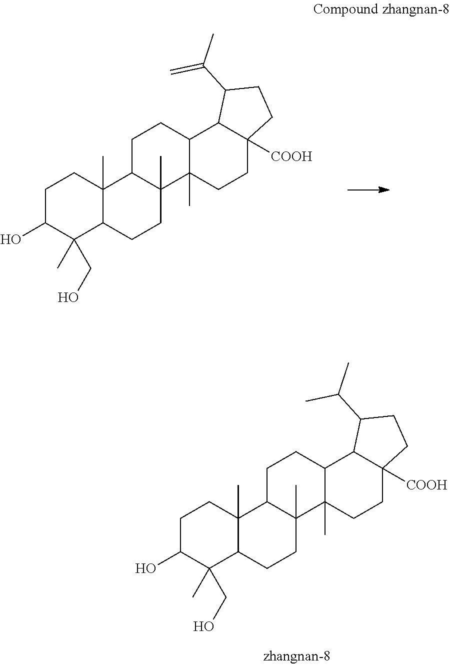

H-NMR (400 MHz, CDCl3) δ: 0.727 (3H, d, J=6.4 Hz, CH3), 0.816 (3H, d, J=6.8 Hz, CH3), 0.790, 0.853, 0.910, 1.289 (each 3H, s, 4×CH3), 2.243 (4H, m)_{∘}

### C-NMR (400 MHz, CD3OD) δ: 70.58, 64.82, 56.33, 50.39, 48.98, 47.09, 44.37, 42.68, 42.49, 39.38, 38.76, 37.64, 36.90, 34.26, 29.96, 27.37, 27.18, 23.62, 23.23, 21.17, 17.99, 16.81, 16.66, 15.22, 14.78, 12.89_{∘}

### MS(ESI): m/z 444 [M−2OH]⁻_{∘}

The preparation methods of the three new derivatives of 23-hydroxyl-betulinic acid, i.e., 3-carboxyl-23-hydroxyl-betulinic acid, 3-carboxyl-23-hydroxyl-dihydrobetulinic acid, and 23-hydroxyl-dihydrobetulinic acid can refer to Hashimoto F, Kashiwada Y, Cosentino L M, Chen C H, Garrett P E, Lee K H., Anti-AIDS agents-XXVII. Synthesis and anti-HIV activity of betulinic acid and dihydrobetulinic acid derivatives. Bioorg Med Chem. 1997 December; 5 (12):2133-43. These three new compounds can widely inhibit growth of various types of human tumor cell lines, and further induce tumor cells apoptosis. In the research of human tumor xenograft models, 3-carboxyl-23-hydroxyl-betulinic acid is proved to be an effective antitumor agent, and can powerfully inhibit growth of human melanoma, leukemia, malignant glioma, prostate cancer, lung cancer, and colon cancer cells in the body. 3-carboxyl-23-hydroxyl-betulinic acid has great potential to be developed as a new antineoplastic drug of wide spectrum. The present invention also discovers that 3-carboxyl-23-hydroxyl-betulinic acid, 3-carboxyl-23-hydroxyl-dihydrobetulinic acid and 23-hydroxyl-dihydrobetulinic acid also have effects of anti-HIV/AIDS, in which 3-carboxyl-23-hydroxyl-dihydrobetulinic acid is particularly strong against HIV virus and is stronger than the clinically used positive control groups of anti-AIDS drugs. Thus, 3-carboxyl-23-hydroxyl-dihydrobetulinic acid has a great potential to be developed as a new anti-AIDS drug.

### BRIEF DESCRIPTION OF DRAWINGS

FIG. 1 is a plot of tumor volume of tumor cell—days after transplant, when using compound zhangnan-5 or control group to treat human prostate cancer (LnCAP) that is subcutaneous inoculated.

FIG. 2 is a plot of tumor volume of tumor cell—days after transplant, when using compound zhangnan-5 or control group to treat human lung cancer (NCI-H23) that is subcutaneous inoculated.

FIG. 3 is a plot of tumor volume of tumor cell—days after transplant, when using compound zhangnan-5 or control group to treat human colon cancer (HCT-116) that is subcutaneous inoculated.

FIG. 4 is a plot of tumor volume of tumor cell—days after transplant, when using compound zhangnan-5 or control group to treat human melanoma (G-361) that is subcutaneous inoculated. FIG. 5 is a plot of tumor volume of tumor cell—days after transplant, when using compound zhangnan-5 or control group to treat human malignant glioma (U-251) that is subcutaneous inoculated.

### DETAILED DESCRIPTION OF PREFERRED EMBODIMENTS

The present invention will be described in connection with the drawings.

The present invention relates to three new derivatives of 23-hydroxyl-betulinic acid, i.e., compounds zhangnan-5, zhangnan-7 and zhangnan-8. Their chemical names, chemical formula, various parameters characterizing chemical structures and preparation methods have been disclosed in the Summary. The biological activities of these three new compounds will be illustrated herein through experiments.

Compounds zhangnan-5, zhangnan-7 and zhangnan-8 can widely inhibit human tumor cell strain but have no effect to normal cell strain: (see Carmichael J, DeGraff W G, Gazadar A F, Minna J D, Mitchell J B; Evaluation of a tetrazolium-based semiautomated colormeteric assay: assessment of chemosensitivity testing. Cancer Research, 1987, 47: 943-946).

We use human tumor cell lines to conduct cytotoxicity analysis. Thirty-one (31) kinds of human tumor cell strains are cultivated in the normal culture medium containing various compounds of zhangnan-5, zhangnan-7 and zhangnan-8. The human tumor lines are bought from ATCC and NCI, and is cultivated with DMEM culture solution containing 10% FBS in 370C couveuse containing 5% CO₂. The merged cells are digested by trypsinase, and are counted after washed by the culture solution. 3000˜6000 cells are added into each hole of the 96-hole culture plate, and are hatched for 16 hours or 24 hours. Then, compounds zhangnan-5, zhangnan-7 and zhangnan-8 with different concentrations are added into the holes. Upon further cultivated for another 72 hours, MTT tests are conducted to cells in group treated with drugs and the control group cells, to determine the survival condition of the cells. The results are shown in Table 1. Table 1 shows that compounds zhangnan-5, zhangnan-7 and zhangnan-8 can widely inhibit human tumor cell strains, in which most human body tumor cells are especially sensitive to compound zhangnan-5. EC50 of some tumor cells to this compound is below 0.1 μm. However, the normal human epithelium of mammary gland (MCF-10a) and normal mice fibroblast (MEF) are not sensitive to compounds zhangnan-5, zhangnan-7 and zhangnan-8. Even if the concentration of the compounds is up to 30 μm, the cells can still grow well.

TABLE 1
Compounds zhangnan-5, zhangnan-7 and zhangnan-8
inhibit human tumor cell growth in vitro

| | | | Zhangnan- | Zhangnan- |
|---|---|---|---|---|
| | | | 7 | 8 |
| Human Tumor | | Zhangnan-5 | EC50 | EC50 |
| Cell Strain | Cell Type | EC50 (μM) | (μM) | (μM) |
| LnCAP | Prostate | 0.69 | 15.6 | 7.8 |
| D145 | Prostate | 0.85 | 14.8 | 8.4 |
| PC3 | Prostate | 0.91 | 16.2 | 8.9 |
| HCT-116 | Colon | 0.09 | 15.1 | 7.8 |
| Widr | Colon | 0.28 | 14.8 | 8.1 |
| HT29 | Colon | 0.47 | 13.9 | 8.9 |
| LoVo | Colon | 0.15 | 15.2 | 9.6 |
| CCL-225 | Colon | 0.23 | 16.1 | 8.6 |
| CCL-247 | Colo | 0.52 | 15.6 | 8.2 |
| NCI-H23 | Lung | 0.32 | 12.8 | 6.7 |
| A549 | Lung | 0.54 | 13.1 | 6.9 |
| MDA-MB-231 | Mammary Gland | 0.47 | 17.5 | 7.2 |
| MDA-MB-435 | Mammary Gland | 0.55 | 19.3 | 7.9 |
| AU-565 | Mammary Gland | 0.59 | 18.2 | 6.8 |
| BT-549 | Mammary Gland | 0.67 | 17.4 | 7.4 |
| MCF-7 | Mammary Gland | 0.58 | 16.9 | 7.8 |
| Caki-1 | Kidney | 0.98 | 20.1 | 9.2 |
| ACHN | Kidney | 1.2 | 18.3 | 8.9 |
| 786-O | Kidney | 3.1 | 16.8 | 8.1 |
| SN12C | Kidney | 1.3 | 17.5 | 7.6 |
| SKOV3 | Ovary | 1.5 | 16.9 | 6.9 |
| IGROV1 | Ovary | 2.1 | 17.4 | 7.2 |
| Mid PaCa-2 | Pancreatic Gland | 0.93 | 18.9 | 9.4 |
| U-251 | Malignant | 0.089 | 17.8 | 5.6 |
| | Glioma | | | |
| SK-MEL-5 | Skin (melanoma) | 0.093 | 18.5 | 4.9 |
| G-361 | Skin (melanoma) | 0.087 | 19.3 | 5.3 |
| MCF-10a | Normal | >30 | >30 | >30 |
| | Mammary Gland | | | |
| | Epithelial Cell | | | |
| SGC-7901 | Stomach | 0.89 | 18.5 | 8.9 |
| EC109 | Esophagus | 0.78 | 17.9 | 9.5 |
| CNE-2Z | Nasopharynx | 1.3 | 19.2 | 10.2 |
| Raji | Lymphoma | 0.098 | 20.5 | 6.9 |
| Jurkat | Leukemia T Cell | 0.14 | 21.3 | 7.1 |
| MEF | Normal Mouse | >30 | >30 | >30 |
| | Fibroblast | | | |

The compounds zhangnan-5, zhangnan-7 and zhangnan-8 and the salts of various forms thereof can be used as drug substance of anti-tumor drugs. The formed salts are produced from reaction of derivatives and alkali. Alkali can be any forms of alkali, which can be inorganic alkali such as sodium hydroxide, potassium hydroxide and the like, and can also be organic alkali such as alkali metal alkoxide and the like. Oral agent or injection agent of anti-tumor drugs can be made of the compounds zhangnan-5, zhangnan-7 and zhangnan-8 or the salts in various forms thereof with drug carrier and/or excipient including corn oil, sodium carboxymethylcellulose and the like.

(2) Toxicity of compounds zhangnan-5, zhangnan-7 and zhangnan-8 to C8166 and PBMC cells:

C8166 (human T cell line) and PBM (using lymphocyte isolation liquid to separate from normal human peripheral blood, and adhered mononuclear cell separated from PBMC) cells in exponential phase are taken. The cells are resuspended after centrifugal (1000 r•min-1, 10 min), and the concentrations are adjusted to 4×105/ml and 2×106/ml. The test samples are double diluted on the 96-hole cell culture plate. In total 6 gradients are set, each having 3 duplicate holes. In addition, positive control group and cell control group are set. Upon adding cell suspended liquid, the test samples are placed in incubator of 370C and 5% CO₂ for incubation. On the seventh (7^{th}) day, MTT method is used to determine toxicity of the samples to cells. The inhibition ratio of the tested samples to the cell growth is calculated according to the following formula, and the value of IC50 (50% inhibition concentration) is calculated.

Inhibition ratio of cell growth (%)=(1−experimental hole OD value/control hole (OD value)×100%

The IC50 values of 23-hydroxyl-betulinic acid and compounds zhangnan-5, zhangnan-7 and zhangnan-8 to C8166 cell are 15.1±3.57 uM, 2.3±0.47 uM, 25±5.2 uM and 10.3±2.9 uM, respectively, while positive control AZT has toxicity to 50% of C8166 cells at 3.437 mM. The IC50 values of 23-hydroxyl-betulinic acid and compounds zhangnan-5, zhangnan-7 and zhangnan-8 to normal person PBMC are 14.9±3.46 uM, 2.4±0.38 uM, 25.8±4.9 uM and 11.6±3.5 uM, respectively. The IC50 of the positive control drug SCH-C (antiretroviral drug for treating AIDS, is antagonist of CCR, and can only prevent R-type virus to combine with cells and have no effect to x-type virus. The molecular weight (MW) is 557. 184.1 mg SCH-C is dissolved into 3.3 ml DMSO, to obtain 100 ummol stock solution, which is stored at 4° C. or −20° C.) to PBMC is 82.667 ummol.

(3) The inhibition effects of compounds zhangnan-5, zhangnan-7 and zhangnan-8 to virus HIV-1IIIB inducing C8166 cell to form syncytium: (see JIANG, Haiou; WANG, Xu; L I, Rurun; CHEN, Dagang; B I, Kunlong, Experimental Research on Recipe Sanhuangsan Capsule for Resisting AIDS virus in Vitro, [J]. Shandong Journal of Traditional Chinese medicine, 2005.8 (1):21-26).

The test sample is double diluted with culture medium on the 96-hole flat culture plate. There are 8 dilutions in total. Each hole 100 ul has 2 duplicate holes. In addition, there are positive control group, cell control group and virus control group. 80 ul C8166 cell of 4×105/ml is added into each hole, and then 20 ul virus HIV-1IIIB supernatant is added into each hole. The final volume of each hole is 200 ul. The sample is placed in incubator of 370C, 5% CO₂ for cultivation. On the third day after infection, the effects of compounds zhangnan-5, zhangnan-7 and zhangnan-8 on syncytium will be observed. Under the inverted microscope, the number of syncytium formed by virus HIV-1IIIB inducing C8166 cell is counted. EC50 is the concentration of compounds zhangnan-5, zhangnan-7 and zhangnan-8 when the compounds inhibit the syncytium to reach 50%. The following formula is used to calculate the inhibition ratio of the sample to syncytium.

Inhibition ratio formed by syncytium (%)=(1−experimental hole syncytium number/control hole syncytium number)×100%

The positive control AZT shows effectively inhibiting HIV-1 inducing C8166 cell to form syncytium. Its EC50 is 1.39 uM, and therapeutic index TI value is 2473. The EC50 values of 23-hydroxyl-betulinic acid and compounds zhangnan-5, zhangnan-7 and zhangnan-8 are 1.2±0.23 uM, 0.5±0.07 uM, 0.0019±0.0002 uM, and 1.1±0.09 uM, respectively; and the therapeutic index are 13, 4.6, 13158 and 9.4, respectively. The inhibiting effect of compounds zhangnan-5, zhangnan-7 and zhangnan-8 on HIV-1IIIB inducing syncytium formation increases with increasing concentration. Comparing with the positive control drug AZT, the therapeutic index (TI) of zhangnan-7 is the highest, which shows significant inhibiting effect to the formation of syncytium.

(4) Quantitative Enzyme Linked Immunoadsorbent method (EIISA) detecting inhibiting effects of compounds to virus HIV-1Ada-Mp24 expression: (see JIANG, Haiou; WANG, Xu; L I, Rurun; CHEN, Dagang; B I, Kunlong, Experimental Research on Recipe Sanhuangsan Capsule Resisting AIDS Virus in Vitro, [J], Shandong Journal of Traditional Chinese medicine, 2005.8 (1):21-26).

Multi-heads sample injector is used to conduct triple dilution to the sample on the 96-hole culture plate. There are 6 dilutions, in which each dilution has 3 duplicate holes, and each hole is 100 ul. In addition, there are positive control group SCH-C (antiretroviral drug for treating HIV/AIDS, is antagonist of CCR, can only prevent type R virus to combine with cells, and have no effect on type x virus. Molecular weight (MW) is 557. 184.1 mg SCH-C is dissolved into 3.3 ml DMSO to obtain 100 ummol stock solution, which is stored at 4° C. or −20° C.), virus control group and cell+virus control group. On the 96-hole flat culture plate, each hole is added with 25 ul virus stock solution containing 1000˜5000 pg/well P24 antigen, which is sufficiently blended. 75 ul mononuclear cell PBMC suspension liquid is added (3×105 cell numbers/hole). Upon placing in incubator of 37° C., 5% CO₂ to the third day, 100 ul of culture medium is added to each hole. On the 7^{th} day, cell culture supernatant is collected for p24 antigen test. According to p24 kit description, ELISA method is used to measure the virus HIV-1 p24 antigen concentration of each sample cell culture hole supernatant. The following formula is used to calculate the inhibition ratio of sample to virus.

Inhibition ratio of sample to virus p24 antigen (%)=(1−experimental hole OD value/cell+virus control hole OD value)×100%. Reed and Mueneh method is used to calculate EC50 of the test sample, and to further calculate the therapeutic index (TI) (TI=IC50/EC50). When using the kit to determine the content of HIVp24 antigen, it is found that the added viral load (V) is 896.3 pg/ml, while the virus p24 content of virus multiplication control (virus+cell, V+C) is 1452.9 pg/ml. The positive control SCH-C, 23-hydroxyl-betulinic acid, compounds zhangnan-5, zhangnan-7 and zhangnan-8 all reach an inhibition ratio above 50% within the tested range. The positive control SCH-C inhibits HIV-1Ada-M p24 antigen expression. Its EC50 is 0.032 ummol, and therapeutic index is 2583. The EC50 values of 23-hydroxyl-betulinic acid, compounds zhangnan-5, zhangnan-7 and zhangnan-8 are 1.9±0.39 uM, 0.7±0.19 uM, 0.0026±0.0005 uM, and 1.6±0.23 uM, respectively; and the therapeutic index are 7.8, 3.4, 9923 and 7.3, respectively. 23-hydroxyl-betulinic acid, compounds zhangnan-5, zhangnan-7 and zhangnan-8 all have inhibition effect to HIV-1Ada-M, in which zhangnan-7 has the strongest inhibition activity to HIV-1Ada-M, and is significantly stronger than positive control compound SCH-C. In addition, the inhibition effect to HIV-1Ada-M has significant dose-effect relationship with its concentration.

The compounds zhangnan-5, zhangnan-7 and zhangnan-8 and the salts of various forms thereof can be used as drug substance of anti-AIDS drugs. The formed salts are produced from reaction of derivatives and alkali. Alkali can be any forms of alkali, which can be inorganic alkali such as sodium hydroxide, potassium hydroxide and the like, and can also be organic alkali such as alkali metal alkoxide and the like. Oral agent or injection agent of anti-AIDS drugs can be made of the compounds zhangnan-5, zhangnan-7 and zhangnan-8 or the salts in various forms thereof with drug carrier and/or excipient including corn oil, sodium carboxymethylcellulose and the like.

(5) Pharmacokinetics research of the compound zhangnan-5 in mice body: (see Singh S S, Shah H, Gupta S, Jain M, Sharma K, Thakkar P, Shah R. Liquid chromatography—electrospray ionisation mass spectrometry method for the determination of escitalopram in human plasma and its application in bioequivalence study. J Chromatogr B Analyt Technol Biomed Life Sci. 2004 Nov. 25; 811 (2):209-15).

Four 8-week old BlebC mice (two male and two female) are used, feeding with the compound zhangnan-5 of 100 mg/kg for single dose. At 0.5, 1, 3, 6, 12, 24, 48, and 72 hours upon mouth feeding the drug, blood is collected via vena ophthalmica and blood plasma is made, to determine the plasma concentration of the given compound zhangnan-5.

TABLE 2
pharmacokinetics parameter of compound zhangnan-5 in mice body

| Pharmacokinetics parameter | average number +/− standard deviation |
|---|---|
| T½ (hour) | 11.5 +/− 0.8 |
| Tmax (min) | 85 +/− 7 |
| Cmax (μM) | 65 +/− 9 |

(6) Research on the acute toxicity of compound zhangnan-5 to mice upon feeding the drug: (see Gol'dberg L E, Stepanova E S, Vertogradova T P, Shevniuk L A, Shepelevtseva G. Preclinical toxicological study of the new antibiotic eremomycin. Its acute toxicity for laboratory animals, Antibiot Med Biotekhnol. 1987, 32 (12): 910-5).

Two groups of 8-week old BlebC mice are taken, and each group has 10 mice (five male and five female). The mice are fed with the compound zhangnan-5 (prepared with corn oil or carrier) in single dose 500 mg/kg and in multi-dose 200 mg/kg (QDX15), respectively. Then, observation is made to the mice for 1 week and 4 week, respectively. The mice are weighted every other day. When the test is over, the tested mice are killed for pathological analysis. The results show that no toxicity is observed in the mice feeding the compound zhangnan-5 in single dose 500 mg/kg and in multi-dose 200 mg/kg (QDX15), and all of the tested mice grow well with no single death. Thus, the compound zhangnan-5 has great potential to be developed as a new antineoplastic drug of wide spectrum. The compound zhangnan-5 can be used as drug substance of anti-tumor drugs, preferably used as drug substance of anti-malignant solid tumor drugs. The compound zhangnan-5 can also be used with drug carrier and/or excipient to make anti-tumor drug. The drug carrier and/or excipient can be, for example, corn oil, sodium carboxymethylcellulose, and the like.

Typically, the recommended oral dosage of compound zhangnan-5 is 165 mg/m² (body surface area) every day, for 3 weeks, and rest for a week as one course of treatment. The total dose per day of compound zhangnan-5 is taken once orally after half hour of breakfast, and can be adjusted by physician based on actual condition.

(7) Drug administration of human tumor xenograft model and nude mice tumor transplantation model: (see Harrison S D Jr; Growth of human tumor cells in athymic mice; J Natl Cancer Inst. 1991 Oct. 16; 83 (20): 1509).

T cell deficiency nude mice (nu/nu) (male, 6-week old) are bought from Charles River Lab, and are raised in pathogen-free environment according to guidelines of animal raising and using committee of university. The number of 5×106 LNCaP, NCI-H23, HCT-116, G-361 and U-251 cells are suspended in 0.2 ml HBSS or matrigel (50: 50, v/v), and are subcutaneously vaccinated to the side abdomen region of the mice. When the average diameters of the tumors are 7˜8 mm, the mice having tumor size of 100˜200 mm3 are chosen, and are given with treatment group of compound zhangnan-5 prepared with corn oil and are only given with control group of empty carrier (corn oil), respectively. In order to ensure that the compound zhangnan-5 treatment group and control group have substantially equal tumor volume distribution at the beginning of treatment, the mice are divided into three categories: small tumor volume (length<4 mm), medium tumor volume (4˜8 mm), and large tumor volume (>8 mm). The number of mice from same categories in the control group and the compound zhangnan-5 treatment group are approximately same.

In the oral feeding test, the compound zhangnan-5 (30 mg/kg) is dissolved in corn oil. Each day the mice are tube fed with 200 μl drug liquid in single dose (prepared with corn oil, containing 0.75 mg compound zhangnan-5) for 5 times a week and for 3 weeks. The control group is given placebo (corn oil) according to same method. The mice are raised separately, and are allowed to self-help feeding.

Evaluation of Anti-Tumor Effect:

The tumor is measured in two vertical directions every 3˜4 days. The tumor volume is calculated based on the following formula:

V=(a2×b)/2

Wherein, a is the tumor width (smaller diameter), and b is the length (larger diameter). The relative tumor volume (RTV) of each tumor is defined as the ratio of the tumor volume at specific time point and the tumor volume at the beginning of treatment. Mean value is calculated for each treatment group. The tumor growth inhibition value (TGI) is calculated based on the following formula, to determine the anti-tumor activity:

TGI(%)=T/C×100

wherein T is the mean value of RTV at the end point (4 weeks) of the test for the treatment group, and C is the mean value of RTV at the end point of the test for the control group. The minimum anti-tumor activity standard of US National Cancer Institute is applied (T/C≦42%).

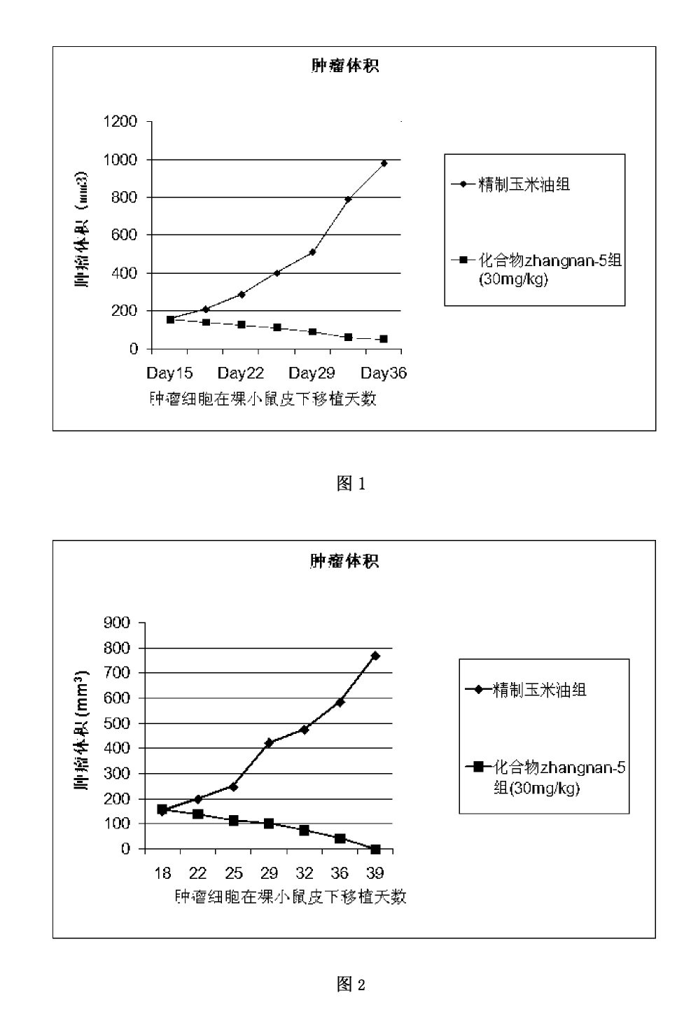

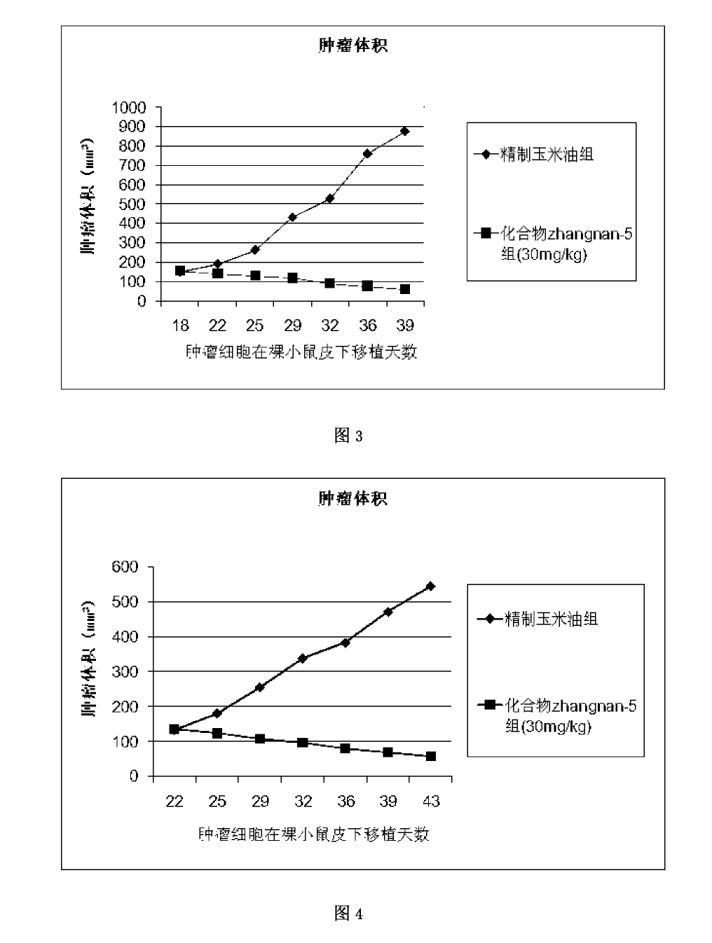

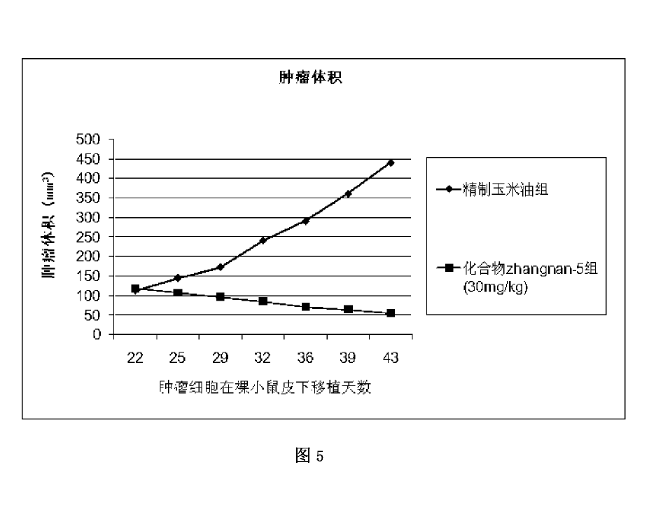

On the basis that the compound zhangnan-5 can effectively inhibit proliferation of many kinds of tumor cells in vitro, human tumor is transplanted to nude mice (via subcutaneous vaccination), to evaluate the anti-tumor effect of the compound zhangnan-5. The tested tumor cells include human prostate cancer cell LnCAP, human lung cancer cell NCI-H23, human colon cancer cell HCT-116, human melanoma cell G-361 and human malignant glioma cell U-251. The results are shown in FIGS. 1-5. The compound zhangnan-5 can powerfully inhibit growth of the three tumors, and the TGI values are 5%, 0%, 6.9%, 10.8%, and 12.5%, respectively. In addition, no obvious side effects have been found for zhangnan-5, including changes in weight. Such data clearly indicates that the compound zhangnan-5 is an excellent candidate compound for developing new anti-tumor drug.

1. A 23-hydroxyl-betulinic acid derivative, having a chemical name 3-carboxyl-23-hydroxyl-betulinic acid, and a chemical formula:

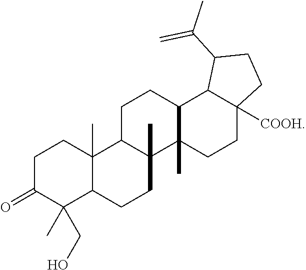

2. A method for preparing the 23-hydroxyl-betulinic acid derivative of claim 1, comprising:
(1) dissolving 23-hydroxyl-betulinic acid in pyridine, stirring, slowly adding acetic anhydride dropwise and stirring, then slowly adding saturated ammonia chloride solution dropwise until no bubbles are generated, extracting with ethyl acetate to obtain organic lavers and merging the organic layers, using a saturated sodium chloride solution to wash the organic layers, and then drying with anhydrous sodium sulfate, spinning dry the ethyl acetate organic solvent and then conducting a column separation to obtain compounds 3,23-diacetyl-23-hydroxyl-betulinic acid, 23-diacetyl-23-hydroxyl-betulinic acid, and 3-diacetyl-23-hydroxyl-betulinic acid
(2) dissolving and dispersing 23-diacetyl-23-hydroxyl-betulinic acid in methylene chloride, adding oxygenant dicyclohexylcarbodiimide in an ice-bath, stirring at room temperature, spinning dry the methylene chloride solvent, dissolving in ethyl acetate and conducting a column separation with a coarse silica gel to obtain 3-carboxyl-23-diacetyl-23-hydroxyl-betulinic acid; and
(3) dissolving and dispersing 3-carboxyl-23-diacetyl-23-hydroxyl-betulinic acid in methanol, adding a saturated potassium carbonate solution, stirring the reaction at room temperature, extracting with ethyl acetate to obtain organic layers and merging the organic layers, using a saturated sodium chloride solution to wash the organic layers, and then drying with anhydrous sodium sulfate, spinning dry the ethyl acetate organic solvent and conducting a column separation to obtain 3-carboxyl-23-hydroxyl-betulinic acid.

3. A 23-hydroxyl-betulinic acid derivative, having a chemical name 3-carboxyl-23-hydroxyl-dihydrobetulinic acid, and a chemical formula:

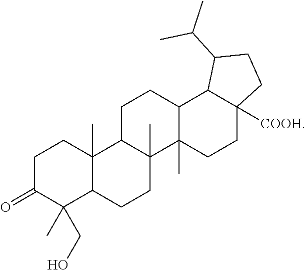

4. A method for preparing the 23-hydroxyl-betulinic acid derivative of claim 3, the method comprising:
dispersing and dissolving 3-carboxyl-23-hydroxyl-betulinic acid in methanol, adding reductant Pd/C and stirring in a vacuum under a flow of hydrogen at room temperature, filtering out Pd/C, and then spinning dry the methanol solvent to obtain a 3-carboxyl-23-hydroxyl-dihydrobetulinic acid.

5. A 23-hydroxyl-betulinic acid derivative, having a chemical name 23-hydroxyl-dihydrobetulinic acid, and a chemical formula:

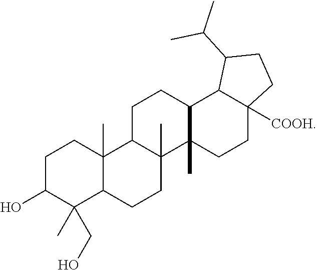

6. A method for preparing the 23-hydroxyl-betulinic acid derivative of claim 5, the method comprising:
dispersing and dissolving the 23-hydroxyl-betulinic acid in methanol, adding reductant Pd/C and stirring under a flow of hydrogen at room temperature, filtering out the Pd/C, and then spinning dry the methanol solvent to obtain 23-hydroxyl-dihydrobetulinic acid.

7. Use of the 23-hydroxyl-betulinic acid derivative according to claim 1 or its salts in various forms in preparing an anti-tumor drug.

8. Use of the 23-hydroxyl-betulinic acid derivative according to claim 1 or its salts in various forms in preparing anti-AIDS drug.

9. A pharmaceutical composition comprising the 23-hydroxyl-betulinic acid derivative according to claim 1 or its salts in various forms, and a suitable drug carrier and/or excipient.

10. The pharmaceutical composition according to claim 9, wherein the pharmaceutical composition comprises an oral agent or injection agent, and wherein the drug carrier and/or excipient includes corn oil, and sodium carboxymethylcellulose.

11. A pharmaceutical composition comprising the 23-hydroxyl-betulinic acid derivative according to claim 3 or its salts in various forms, and a suitable drug carrier and/or excipient.

12. A pharmaceutical composition comprising the 23-hydroxyl-betulinic acid derivative according to claim 5 or its salts in various forms, and a suitable drug carrier and/or excipient.

13. Use of the 23-hydroxyl-betulinic acid derivative according to claim 3 or its salts in various forms in preparing anti-AIDS drug.

14. Use of the 23-hydroxyl-betulinic acid derivative according to claim 5 or its salts in various forms in preparing anti-AIDS drug.

15. Use of the 23-hydroxyl-betulinic acid derivative according to claim 3 or its salts in various forms in preparing an anti-tumor drug.

16. Use of the 23-hydroxyl-betulinic acid derivative according to claim 5 or its salts in various forms in preparing an anti-tumor drug.
